(19) United States
(12) Patent Application Publication
(10) Pub. No.: US 2023/0180809 A1
(43) Pub. Date: Jun. 15, 2023

(54) NUTRACEUTIC COMPOSITION COMPRISING LIMONENE AND A DRY EXTRACT OF COCOA FIBRE FOR THE TREATMENT OF OBESITY

(71) Applicant: TARGETING GUT DISEASE SRL, Bologna (BO) (IT)

(72) Inventor: Maria Chiara VALERII, Bologna (BO) (IT)

(21) Appl. No.: 17/925,437

(22) PCT Filed: May 31, 2021
(86) PCT No.: PCT/EP2021/064568

§ 371 Date: Nov. 15, 2022

(30) Foreign Application Priority Data
Jun. 5, 2020 (EP) 20178426.1

(51) Int. Cl. A23L 33/105; A23L 33/22

(52) U.S. Cl. CPC A23L 33/105; A23L 33/22

(57) ABSTRACT

A nutraceutical composition comprising natural or synthetic limonene or an essential oil having a content of limonene of at least 30% by weight and cocoa fibre is described for human or veterinary use in the treatment of obesity.

### DESCRIPTION

The present invention relates to a nutraceutical composition comprising essential oils and cocoa fibre, more in particular to a nutraceutical composition comprising natural or synthetic limonene or an essential oil having a content of limonene of at least 30% by weight and cocoa fibre for human or veterinary therapeutic use in the treatment of obesity.

Essential oils and their active ingredients have particularly attractive biological activities especially in the case of oral consumption.

Conveniently dosed, they can be used for their slimming, germicidal, fungicidal, antibacterial, anti-inflammatory, anti-parasite and antibiotic activities (Li et al, 2013, EJP, 716; 46-55; Thapa et al., 2012, Microbiology, 158 (11); 2870-2877). However, these substances present enormous problems in therapeutic applications due both to their aggressiveness with regard to the oral, oesophagus, gastric and intestinal mucus and their toxic nature.

Even using correct therapeutic doses, extracts of vegetable origin that contain essential oils and/or their components (including synthetic analogues) may cause inflammations, irritations, erosions or other damages to the mucus with which they come into contact, such as for example in the mouth.

Moreover, if for example, a therapeutic action of essential oils is sought, it is very difficult to carry the corresponding active ingredients to the streches of interest without damaging, even in a significant manner, the intestinal tracts passed through before reaching the area of interest. Even the fast absorption of essential oils, especially in the upper intestinal tract (called small intestine), can prevent them from effectively reaching the lower tracts, such as colon for example, where they would exert their therapeutic action topically. Moreover, in some diseases, it would be recommended to have both a topical and systemic effect of the active components that should act in the digestive tract and in the bloodstream on circulating molecules.

The combination of the topical and systemic effect is essential in the treatment of obesity with natural or synthetic limonene or with essential oils containing limonene, in particular with orange essential oil.

Orange essential oils and their components, in particular limonene, are known to have an anti-obesity effect (Dhu Li et al., Bioscience, Biotechnology, and Biochemistry, vol. 83(5), 2019, 923-932).

The patent application EP 3 097 921 filed in the name of the Applicant discloses a composition comprising essential oils and a dry plant extract, root, sprout or fruit extract that allows to overcome the above-mentioned drawbacks and to administer essential oils or components thereof so that they exert their topical action in the intestine without damaging the mucosa and organs of the digestive tract.

Although particularly advantageous and innovative for administering essential oils or components thereof, the composition disclosed in EP 3 097 921 comprising dry extract, in particular dry extract of glucomannan, ginger root, turmeric root, boswellia, cabbage and fabaceae seeds, does not guarantee also an effective systemic effect in case of administration of essential oils or components thereof for treating obesity. The Application has now found that the dual local and systemic effect is obtained by using cocoa fibre instead of dry plant extracts, root, sprout or fruit extracts disclosed in EP 3 097 921.

The object of the present invention is therefore a nutraceutical composition comprising natural or synthetic limonene or an essential oil having a content of limonene of at least 30% by weight and cocoa fibre.

The composition according to this invention allows to cancel or significantly reduce the negative effects of the essential oil or components thereof on mucus, to partially release the active ingredient in the first part of the digestive tract so that it is absorbed and that another part of the active ingredient can reach the other parts of the digestive tract and locally exert its action.

The composition according to this invention, thanks to its peculiar dual local and systemic effect due to the use of cocoa fibre, is particularly suitable for the treatment of obesity.

In the composition according to this invention, a preferred essential oil is a citrus essential oil, in particular orange essential oil.

Non-limiting esamples of essential oils having a content of limonene of at least 30% by weight that can be used according to the present invention are the products marketed as “O.E. Arancio Sicilia”, O.E. Arancio Brasile” and Natural Terpenes 100% from Industrie Chimiche Muller & Koster SpA.

According to a particularly preferred embodiment, the composition of the present invention comprises natural or synthetic limonene.

In the present description, the essential oil and the limonene can also be generically indicated as active ingredient.

Preferably, the active ingredient is present in the composition in a quantity comprised between 5% and 50% by weight, more preferably in a quantity comprised between 10% and 25% by weight.

The composition according to this invention is characterized by the fact that the active ingredient is mixed with cocoa fibre.

In the present description, the term “cocoa fibre” is used to indicate cocoa fibre powder or dry cocoa extract or cocoa bean powder or powder obtained from cocoa bean pericarp. The use of the term “cocoa fibre” also indicates that the main component is natural vegetable fibre.

The cocoa fibre used in the composition of the present invention is available on the market and is generally referred to as “cacao fibra polv.” or “Theobroma cacao, Shell”. Preferably, the cocoa fibre is present in the composition of the invention in a quantity comprised between 50% and 95% by weight, more preferably in a quantity comprised between 75% and 90% by weight.

Preferably, the natural fibre has a grain size comprised in a range of from -1 to +8 of the Krumbein (φ) phi scale (1934).

The compositions of the invention are prepared according to a similar process to that disclosed in the already-mentioned patent application EP 3 097 921 in the name of the Applicant.

Said process provides for mixing the active ingredient with cocoa fibre under continuous stirring in order to form solid spherical lumps of variable dimensions. Said lumps are then subjected to a breaking step by mechanical action, followed preferably by a mixing step until complete dissolution of the lumps. Advantageously, the composition according to the invention can be encapsulated in normal gelatine capsules, designed to contain liquid, solid and/or powder substances. In this way, the compositions of the invention allow the encapsulation of the active ingredient, which is normally volatile, in normal gelatine capsules for oral administration.

The compositions of the present invention are useful in the treatment of obesity for human or veterinary use.

A further object of the present invention is therefore the use of a nutraceutical composition comprising natural or synthetic limonene or an essential oil having a content of limonene of at least 30% by weight and cocoa fibre for human or veterinary therapeutic use in the treatment of obesity.

In one embodiment, the composition for use in the treatment of obesity comprises also at least one pharmaceutically acceptable carrier that allows its formulation in conventional dosage forms, such as for example tablets, granules, capsules or suspensions for oral administration.

The preparation of these dosage forms and the selection of the pharmaceutically acceptable carriers are comprised within the knowledge of the skilled person.

The dose of the compositions according to the invention for human or veterinary use is preferably comprised between 5 mg and 10 g/day of composition, for example on the basis of the species, means of administration, age and weight of the subject.

By way of example and without limiting the scope of the invention, preferred embodiments of the composition according to the invention are described below.

### Example 1

Composition comprising:
natural or synthetic limonene;
cocoa fibre powder or dry cocoa extract or cocoa bean powder or powder obtained from cocoa bean pericarp.
60 g of cocoa fibre are added to 10 g of natural or synthetic limonene.

After the addition of cocoa fibre to the natural or synthetic limonene, under continuous stirring, solid spherical lumps of variable dimensions are formed.

The lumps are broken by mechanical action (using for example a vibromixer or similar instrument designed to break the lumps) and the mixture is mixed until complete dissolution of the aggregates.

With reference to the above-mentioned examples, some results are reported below of animal and/or clinical trials supporting the therapeutic use.

Demonstration of the slimming effect according to example 1 in the treatment of obesity.

The composition of example 1 has been administered to a group of mice with obesity induced by a high lipid diet, with a dose of 60 mg/day for a cycle with a duration of 6 weeks.

The effectiveness of the treatment was demonstrated by monitoring the weight of the animals, which were well fed with a weight-gaining diet with a high lipid content. After 6 weeks of treatment, the group of animals treated had an average weight reduced by 12% compared with the group of control animals. The animals treated also showed lower blood sugar and triglyceride levels compared with the control group.

Demonstration of the slimming effect of limonene in obese mice.

Animal feed having a concentration of 0.5% in limonene corresponding to a daily dose of 0.6 g/kg of weight in humans. After 12 weeks of high lipid diet that caused obesity, mice were administered ad libitum, animal feed containing 0.5% of limonene for 2 weeks. The efficacy of the treatment was demonstrated by decreasing the size of both white and brown adipocytes, by reducing the serum triglycerides up to 47% and by preventing the accumulation of lipids in the cells.

The present invention therefore relates to a new mixing of natural or synthetic limonene or of essential oil having a content of limonene of at least 30% by weight, as active ingredient, with cocoa fibre used for the treatment of obesity in human or animals.

The composition is a damp powder (hygroscopic) which releases the active component in the area of interest. The specific cocoa fibre composition allows a partial and gradual release of limonene in the small intestine during transit where it is absorbed and exerts its important action on lipid and glycemic profile in obese individuals. In the large intestine, the oily remaining component, harnessed in fibre, is released when the fibre is degraded by the intestinal bacterial component.

At this moment, the remaining part of the active ingredient modulates the subject metabolism by acting on the intestinal microbiota.

Such a dual effect is obtained by using cocoa fibre but not with other vegetable fibres which convey the active ingredient almost exclusively through the colon but do not allow it to be absorbed.

The fibre also prevents the evaporation of the essential oil component and therefore allows it to be encapsulated in wrappers of any type.

The compositions according to the present invention are particularly advantageous since the obtained mixture is stable and the active component of the mixture (essential oils, components thereof or vegetable origin extracts which they contain, including synthetic analogues) is absorbed on a matrix which eliminates the majority of, or completely, the negative effects on the mucus, allowing a slow and controlled release, in particular along the whole intestinal tract.

The particular cocoa fibre composition allows to release the active partially in the small intestine where it is absorbed and can act systemically, partially in the colon where it can act topically. This characteristic is the basis for the efficacy of the composition.

From a biological point of view, the mixture according to the invention allows the active ingredient to be taken free of the side effects which there would be without mixing. It also allows a slower release of the active ingredient (retard effect), which while starting in the stomach, continues in the small and large intestine where the fibre is demolished by the intestinal microbiota (bacterial flora) and the active ingredient may perform its slimming effect along with its germicidal, fungicidal, antibacterial, anti-inflammatory, anti-parasite and antibiotic actions.

1. A nutraceutical composition comprising natural or synthetic limonene or an essential oil having a content of limonene of at least 30% by weight and cocoa fibre.

2. The composition according to claim 1, wherein the essential oil is orange essential oil.

3. The composition according to claim 2, wherein the cocoa fibre is cocoa fibre powder or dry cocoa extract or cocoa bean powder or powder obtained from cocoa bean pericarp.

4. The composition according to claim 3, wherein the limonene or the essential oil are present in a quantity comprised between 5% and 50% by weight.

5. The composition according to claim 4, wherein the cocoa fibre is present in a quantity comprised between 50% and 95% by weight.

6. The composition according to claim 5, wherein the fibre has a grain size comprised in a range of from -1 to +8 of the Krumbein phi scale.

7. A process for the preparation of the composition according to claim 1, comprising mixing limonene or essential oil with cocoa fibre under continuous stirring in order to form solid spherical lumps of variable dimensions, followed by a breaking step by mechanical action.

8. A process for treating obesity, comprising the step of administering the composition according to claim 1 for human or veterinary therapeutic use in the treatment of obesity.

9. The process according to claim 7, further comprising the step of mixing the limonene or essential oil and cocoa fibre after the breaking step, until complete dissolution of the lumps.

10. The composition according to claim 5, wherein quantity of the cocoa fibre is comprised between 75% and 90% by weight.

11. The composition according to claim 4, wherein the quantity of the limonene or the essential oil is comprised between 10% and 25% by weight.

12. The composition according to claim 1, wherein the cocoa fibre is present in a quantity comprised between 50% and 95% by weight.

13. The composition according to claim 12, wherein quantity of the cocoa fibre is comprised between 75% and 90% by weight.

14. The composition according to claim 1, wherein the limonene or the essential oil are present in a quantity comprised between 5% and 50% by weight.

15. The composition according to claim 14, wherein the quantity of the limonene or the essential oil is comprised between 10% and 25% by weight.

16. The composition according to claim 1, wherein the cocoa fibre is cocoa fibre powder or dry cocoa extract or cocoa bean powder or powder obtained from cocoa bean pericarp.

17. The composition according to claim 1, wherein the cocoa fibre has a grain size comprised in a range of from -1 to +8 of the Krumbein phi scale.
